SEQUENCE LISTING (31 sequences)

SEQ ID NO: 1 - DNA, 689 bp, Homo sapiens
uaaaauuuuuuaauauucuacaauuaacaauuaucucaauuucuuuauucuaaagacauugggauuagaaaaauguucacaagggacuccaaauauugcuguaguauuuguuucuuaaaagaaugauacaaagcagacaugauaaaauauuaaaauuugagagaacuugaugguaaguacauggguguuucuuauuuuaaaauaauuuuucuacuugaaauauuuuacaauacaauaagggaaaaauaaaaaguuauuuaaguuauucauacuuucuucuucuuuucuuuuuugcuauagaaaguauuuauuuuuucuggaacauuuagaaaaaaguuggaucccuaugaacaguggagugaucaagaaauauggaaaguugcagaugagguaaggcugcuaacugaaaugauuuugaaagggguaacucauaccaacacaaauggcugauauagcugacaucauucuacacacuuugugugcauguaugugugugcacaacuuuaaaauggaguacccuaacauaccuggagcaacagguacuuuugacuggaccuaccccuaacugaaaugauuuugaaagagguacucauaccaacacaaaugguugauauggcuaagaucauucuacacacuuugugugcauguauuucugugcacaacuucaaaauggaguacccuaaaauacc

SEQ ID NO: 2 - DNA, 291 bp, Homo sapiens
aaauaauuuuucuacuugaaauauuuuacaauacaauaagggaaaaauaaaaaguuauuuaaguuauucauacuuucuucuucuuuucuuuuuugcuauagaaaguauuuauuuuuucuggaacauuuagaaaaaaguuggaucccuaugaacaguggagugaucaagaaauauggaaaguugcagaugagguaaggcugcuaacugaaaugauuuugaaagggguaacucauaccaacacaaauggcugauauagcugacaucauucuacacacuuugugugcauguaug

SEQ ID NO: 3 - DNA, 90 bp, Homo sapiens
aaaguauuuauuuuuucuggaacauuuagaaaaaaguuggaucccuaugaacaguggagugaucaagaaauauggaaaguugcagaugag

SEQ ID NO: 4 - DNA, 22 bp, Artificial Sequence
  Synthetic
ccaacuuuuuucuaaauguucc

SEQ ID NO: 5 - DNA, 20 bp, Artificial Sequence
  Synthetic
uccaacuuuuuucuaaaugu

SEQ ID NO: 6 - DNA, 22 bp, Artificial Sequence
  Synthetic
ggauccaacuuuuuucuaaaug

SEQ ID NO: 7 - DNA, 18 bp, Artificial Sequence
  Synthetic
gauccaacuuuuuucuaa

SEQ ID NO: 8 - DNA, 21 bp, Artificial Sequence
  Synthetic
cauagggauccaacuuuuuuc

SEQ ID NO: 9 - DNA, 19 bp, Artificial Sequence
  Synthetic
cauagggauccaacuuuuu

SEQ ID NO: 10 - DNA, 20 bp, Artificial Sequence
  Synthetic
aaauaaauacuuucuauagc

SEQ ID NO: 11 - DNA, 20 bp, Artificial Sequence
  Synthetic
aauacuuucuauagcaaaaa

SEQ ID NO: 12 - DNA, 21 bp, Artificial Sequence
  Synthetic
auacuuucuauagcaaaaaag

SEQ ID NO: 13 - DNA, 19 bp, Artificial Sequence
  Synthetic
cagccuuaccucaucugca

SEQ ID NO: 14 - DNA, 19 bp, Artificial Sequence
  Synthetic
caguuagcagccuuaccuc

SEQ ID NO: 15 - DNA, 689 bp, Homo sapiens
uaaaauuuuuuaauauucuacaauuaacaauuaucucaauuucuuuauucuaaagacauugggauuagaaaaauguucacaagggacuccaaauauugcuguaguauuuguuucuuaaaagaaugauacaaagcagacaugauaaaauauuaaaauuugagagaacuugaugguaaguacauggguguuucuuauuuuaaaauaauuuuucuacuugaaauauuuuacaauacaauaagggaaaaauaaaaaguuauuuaaguuauucauacuuucuucuucuuuucuuuuuugcuauagaaaguauuuauuuuuucuggaacauuuagaaaaaacuuggaucccuaugaacaguggagugaucaagaaauauggaaaguugcagaugagguaaggcugcuaacugaaaugauuuugaaagggguaacucauaccaacacaaauggcugauauagcugacaucauucuacacacuuugugugcauguaugugugugcacaacuuuaaaauggaguacccuaacauaccuggagcaacagguacuuuugacuggaccuaccccuaacugaaaugauuuugaaagagguacucauaccaacacaaaugguugauauggcuaagaucauucuacacacuuugugugcauguauuucugugcacaacuucaaaauggaguacccuaaaauacc

SEQ ID NO: 16 - DNA, 756 bp, Homo sapiens
caaaaugggcauuuucaaucuuuuugucauuaguaaaggucagugauaaaggaagucugcaucagggguccaauuccuuauggccaguuucucuauucuguuccaagguuguuugucuccauauaucaacauuggucaggauugaaagugugcaacaagguuugaaugaauaagugaaaaucuuccacuggugacaggauaaaauauuccaaugguuuuuauugaaguacaauacugaauuauguuuauggcaugguaccuauaugucacagaagugaucccaucacuuuuaccuuauaggugggccucuugggaagaacuggaucagggaagaguacuuuguuaucagcuuuuuugagacuacugaacacugaaggagaaauccagaucgauggugugucuugggauucaauaacuuugcaacagugaaggaaagccuuuggagugauaccacaggugagcaaaaggacuuagccagaaaaaaggcaacuaaauuauauuuuuuacugcuauuugauacuuguacucaagaaauucauauuacucugcaaaauauauuuguuaugcauugcugucuuuuuucuccagugcaguuuucucauaggcagaaaagaugucucuaaaaguuuggaauucucaaauucugguuauugaaauguucauagcuuugauaguguuuuucagaagaccaaauuuacagugggagccuugggcuuuuguuuuuuaacagcucuuuuuuguuccugcuucaguggc

SEQ ID NO: 17 - DNA, 356 bp, Homo sapiens
aaaauauuccaaugguuuuuauugaaguacaauacugaauuauguuuauggcaugguaccuauaugucacagaagugaucccaucacuuuuaccuuauaggugggccucuugggaagaacuggaucagggaagaguacuuuguuaucagcuuuuuugagacuacugaacacugaaggagaaauccagaucgauggugugucuugggauucaauaacuuugcaacaguggaggaaagccuuuggagugauaccacaggugagcaaaaggacuuagccagaaaaaaggcaacuaaauuauauuuuuuacugcuauuugauacuuguacucaagaaauucauauuacucugcaaaauau

SEQ ID NO: 18 - DNA, 156 bp, Homo sapiens
gugggccucuugggaagaacuggaucagggaagaguacuuuguuaucagcuuuuuugagacuacugaacacugaaggagaaauccagaucgauggugugucuugggauucaauaacuuugcaacagugaaggaaagccuuuggagugauaccacag

SEQ ID NO: 19 - DNA, 18 bp, Artificial Sequence
  Synthetic
gcuuuccuucacuguugc

SEQ ID NO: 20 - DNA, 18 bp, Artificial Sequence
  Synthetic
cuuuccuucacuguugca

SEQ ID NO: 21 - DNA, 20 bp, Artificial Sequence
  Synthetic
cuuuccuucacuguugcaaa

SEQ ID NO: 22 - DNA, 18 bp, Artificial Sequence
  Synthetic
ggcuuuccuucacuguug

SEQ ID NO: 23 - DNA, 18 bp, Artificial Sequence
  Synthetic
aaggcuuuccuucacugu

SEQ ID NO: 24 - DNA, 20 bp, Artificial Sequence
  Synthetic
ccaaaggcuuuccuucacug

SEQ ID NO: 25 - DNA, 18 bp, Artificial Sequence
  Synthetic
caaaggcuuuccuucacu

SEQ ID NO: 26 - DNA, 19 bp, Artificial Sequence
  Synthetic
uccuucacuguugcaaagu

SEQ ID NO: 27 - DNA, 19 bp, Artificial Sequence
  Synthetic
caagaggcccaccuauaag

SEQ ID NO: 28 - DNA, 20 bp, Artificial Sequence
  Synthetic
ccaccuauaagguaaaagug

SEQ ID NO: 29 - DNA, 19 bp, Artificial Sequence
  Synthetic
ccuuuugcucaccuguggu

SEQ ID NO: 30 - DNA, 18 bp, Artificial Sequence
  Synthetic
cucaccugugguaucacu

SEQ ID NO: 31 - DNA, 756 bp, Homo sapiens
caaaaugggcauuuucaaucuuuuugucauuaguaaaggucagugauaaaggaagucugcaucagggguccaauuccuuauggccaguuucucuauucuguuccaagguuguuugucuccauauaucaacauuggucaggauugaaagugugcaacaagguuugaaugaauaagugaaaaucuuccacuggugacaggauaaaauauuccaaugguuuuuauugaaguacaauacugaauuauguuuauggcaugguaccuauaugucacagaagugaucccaucacuuuuaccuuauaggugggccucuugggaagaacuggaucagggaagaguacuuuguuaucagcuuuuuugagacuacugaacacugaaggagaaauccagaucgauggugugucuugggauucaauaacuuugcaacaguguaggaaagccuuuggagugauaccacaggugagcaaaaggacuuagccagaaaaaaggcaacuaaauuauauuuuuuacugcuauuugauacuuguacucaagaaauucauauuacucugcaaaauauauuuguuaugcauugcugucuuuuuucuccagugcaguuuucucauaggcagaaaagaugucucuaaaaguuuggaauucucaaauucugguuauugaaauguucauagcuuugauaguguuuuucagaagaccaaauuuacagugggagccuugggcuuuuguuuuuuaacagcucuuuuuuguuccugcuucaguggc

(19) United States
(12) Patent Application Publication
(10) Pub. No.: US 2022/0064647 A1
(43) Pub. Date: Mar. 3, 2022

(54) COMPOSITIONS AND METHODS FOR TREATING CYSTIC FIBROSIS

(71) Applicant: SpliSense Ltd., Jerusalem (IL)

(72) Inventors: Yifat OREN, Jerusalem (IL); Ofra BARCHAD-AVITZUR, Jerusalem (IL); Efrat OZERI-GALAI, Jerusalem (IL)

(73) Assignee: SpliSense Ltd., Jerusalem (IL)

(21) Appl. No.: 17/598,274

(22) PCT Filed: Mar. 29, 2020
(86) PCT No.: PCT/IL2020/050381

§ 371 Date: Sep. 26, 2021

Related U.S. Application Data
(60) Provisional application No. 62/825,242, filed on Mar. 28, 2019.

(51) Int. Cl. C12N 15/113; A61K 45/06

(52) U.S. Cl. CPC C12N 15/113; A61K 45/06; C12N 2320/31; C12N 2310/11; C12N 2320/33

(57) ABSTRACT

The present invention is directed to a method for treating cystic fibrosis (CF) using a splicing modulator, such as an antisense oligonucleotide, capable of inducing the skipping of exon 23, exon 24, or both, of the cystic fibrosis transmembrane conductance regulator (CFTR) pre-mRNA. Also provided are a composition and a kit comprising the splicing modulator, and a method of producing thereof.

### CROSS-REFERENCE TO RELATED APPLICATIONS

This application claims the benefit of priority of U.S. Provisional Patent Application No. 62/825,242 titled “COMPOSITIONS AND METHODS FOR TREATING CYSTIC FIBROSIS”, filed Mar. 28, 2019, the contents of which are incorporated herein by reference in their entirety.

### FIELD OF INVENTION

The present invention is in the field of antisense oligonucleotides and therapeutic use of the antisense oligonucleotides.

### BACKGROUND

Cystic fibrosis (CF) is a common, severe autosomal recessive disease caused by mutations in the CFTR gene. The CFTR gene encodes for a chloride channel responsible for chloride transport in epithelial cells. The major manifestations of CF are in the lungs, with more than 90% mortality related to the respiratory disease. The disease in the respiratory tract is linked to the insufficient CFTR function in the airway epithelium.

As of today, approximately 2000 different mutations disrupting the CFTR functions have been identified worldwide, grouped into five distinct classes based on their effect on the CFTR function. Class I includes mutations that lead to non-functional CFTR (large deletions and stop codon mutations). Class II mutations (including the common ΔF508) lead to aberrantly folded CFTR protein that is recognized by the cell quality control mechanism and subsequently degraded, resulting in the absence of mature CFTR protein at the apical cell membrane. Class III mutations lead to full-length CFTR protein being incorporated into the cell membrane, but with defective regulation so that no CFTR function is present. These three classes usually lead to a classic CF phenotype with pancreatic insufficiency, although the severity of lung disease is highly variable. CFTR mutations leading to defective chloride conductance are grouped into Class IV. Class V mutations involve transcription dysregulation, resulting in a decreased amount of otherwise normal CFTR. The latter two classes are often associated with a milder phenotype and pancreatic sufficiency. Specifically, CFTR that results from a class IV mutation inserts into the plasma membrane but exhibits reduced single-channel chloride ion conductance because of reduced chloride permeation and open channel probability.

In recent years, fundamental knowledge of molecular and cellular biology has helped to develop therapies for specific CF mutations/classes of mutations. The current approved therapies include correcting defects in the CFTR protein processing (corrector: VX-809/Lumacaftor, VX-661/Tezacaftor, and VX-445/elexacaftor), chloride channel function (potentiator: VX-770/Kalydeco) and combination of the two. However there is no available therapy for patients carrying other mutations that do not respond to the available therapies (such as stop mutations, missense mutations and more).

Anti-sense oligonucleotides (AOs or ASOs) administration is one of the most promising therapeutic approaches for the treatment of genetic disorders. AOs are short synthetic molecules which can anneal to motifs predicted to be involved in the pre-mRNA splicing. The method is based on splice-switching. The AOs binding to selected sites is expected to mask the targeted region and promote either normal splicing or enable specific exclusion or inclusion of selected exons. AOs are highly specific for their targets and do not affect any other sequence in the cells. Several types of chemically modified AO molecules are commonly used including: 2′-O-methyl-phosphorothioate (2OMP), phosphorodiamidate morpholino oligomer (PMO), peptide nucleic acids (PNAs), 2-methoxyethyl phosphorothioate (MOE), constrained ethyl (cET), Ligand-Conjugated Antisense (LICA) and alternating locked nucleic acids (LNAs).

The AOs modifications maintain their stabilization, improve their target affinity, and provide favorable pharmacokinetic properties and biological stability. The potential of ASOs as therapeutics is demonstrated in several human genetic diseases. Among them is spina muscular atrophy (SMA), in which the inclusion of exon 7 in the gene survival motor neuron 2 (SMN2) leads to a fully functional protein. Based on promising results in studies of neonatal mouse pups with severe SMA, the ASO-based drug SPINRZA® (nusinersen) developed by Biogen and lonis, received FDA approval based on successful completion of a phase-3 clinical trial in patients with infantile-onset SMA, showing a significant improvement in motor function milestones in SMA infants.

### SUMMARY

The present invention is directed to a composition and a method of use thereof comprising oligonucleotides capable of binding to a CFTR pre-mRNA, thereby modulating splicing and restoring or enhancing the function of the CFTR gene product. The present invention thus identifies sequences within the CFTR pre-mRNA which are targeted in order to modulate the splicing cascade of the CFTR pre-mRNA. As demonstrated in the present invention, exclusion of an exon from the CFTR pre-mRNA results in a functional CFTR protein which is produced in sufficient levels by an otherwise aberrant CFTR allele.

The present invention is based, in part, on the finding that artificial “anti-sense” oligonucleotide molecules are able to target and bind predetermined sequences at the pre-mRNA molecule of the CFTR gene, and this binding can modulate the splicing of the pre-mRNA molecule into a mature mRNA which is subsequently translated into a functional CFTR protein in sufficient levels. The targets within a CFTR pre-mRNA molecule are those discovered to be involved in splicing, directly, by affecting their own splicing. The present invention is further based, in part, on the surprising finding that excluding an exon from the CFTR mature protein, renders it partially functional.

According to a first aspect, there is provided a method for treating cystic fibrosis (CF) in a subject in need thereof, comprising administering to the subject a therapeutically effective amount of a synthetic antisense oligonucleotide (ASO), wherein the ASO induces the skipping of exon 23 or exon 24, of the cystic fibrosis transmembrane conductance regulator (CFTR) pre-mRNA, thereby treating CF in the subject, and wherein the ASO targets at least one CF-conferring mutation located in exon 23 or exon 24, of the CFTR pre-mRNA.

According to another aspect, there is provided a composition comprising an ASO comprising 14 to 25 bases having at least 80% complementarity to a CFTR pre-mRNA and characterized by inducing splicing activity of exon 23 or exon 24 of the CFTR pre-mRNA.

A kit comprising: (a) at least one ASO; and at least one of: (b) at least one CFTR modifier; or (c) at least one CF drug, wherein the at least one ASO targets a CF-conferring mutation located in exon 23, exon 24, or both, of CFTR pre-mRNA, and wherein the CFTR modifier is selected from the group consisting of: CFTR potentiator, CFTR corrector, Translational Read-Through agent, and CFTR amplifier.

According to another aspect, there is provided a method for producing a compound suitable for treating CF, the method comprising: obtaining a compound that binds to exon 23 or exon 24 of the CFTR pre-mRNA, assaying the skipping of exon 23 or exon 24 of the CFTR pre-mRNA in the presence of the obtained compound, and selecting at least one compound that induces the exclusion of exon 23 or exon 24 from the CFTR pre-mRNA, thereby producing a compound suitable for treating CF.

In some embodiments, the method further comprises administering to the subject a therapeutically effective amount of one or more CFTR modifiers.

In some embodiments, the CFTR modifier increases the duration of the CFTR gate being open, chloride flow through the CFTR gate, CFTR protein proper folding, the number of CFTR anchored to the cell membrane, or any combination thereof.

In some embodiments, the modifier is selected from the group consisting of: potentiator, corrector, translational read-through agent, and amplifier.

In some embodiments, the modifier is ivacaftor, lumacaftor, tezacaftor, VX-659, VX-445, VX-152, VX-440, or any combination thereof.

In some embodiments, the ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the ASO comprises 14 to 25 bases.

In some embodiments, the ASO comprises 17 to 22 bases.

In some embodiments, the ASO has at least 75% complementarity to: (a) a sequence consisting of: SEQ ID NO: 1, SEQ ID NO: 15, or both; or (b) a sequence consisting of: SEQ ID NO: 16, SEQ ID NO: 31, or both.

In some embodiments, the ASO has at least 75% complementarity to a sequence consisting of SEQ ID NO: 2 or SEQ ID NO: 17.

In some embodiments, the ASO has at least 80% complementarity to: (a) any one of: said SEQ ID NO: 1, said SEQ ID NO: 15, and said SEQ ID NO: 2; or (b) any one of: said SEQ ID NO: 16, said SEQ ID NO: 31, and said SEQ ID NO: 17.

In some embodiments, the ASO has at least 80% complementarity to a sequence consisting of: SEQ ID NO: 3 or SEQ ID NO: 18.

In some embodiments, the ASO comprises 3 mismatched bases at most, compared to a sequence selected from the group consisting of: SEQ ID Nos.: 1-3, 15-18, and 31.

In some embodiments, the one mismatched base at most of the 3 mismatched bases is located not more than 3 bases from the 5′ prime end of the ASO.

In some embodiments, the one mismatched base at most of the 3 mismatched bases is located not more than 3 bases from the 3′ prime end of the ASO.

In some embodiments, the ASO comprises a cytosine complementary to a guanine located at position 336 of the SEQ ID NO: 1, position 136 of the SEQ ID NO: 2, or position 36 of the SEQ ID NO: 3.

In some embodiments, the ASO comprises 4 to 18 nucleotides upstream to the cytosine.

In some embodiments, the ASO comprises:

(SEQ ID NO: 4)
CCAACUUUUUUCUAAAUGUUCC;
(SEQ ID NO: 5)
UCCAACUUUUUUCUAAAUGU;
(SEQ ID NO: 6)
GGAUCCAACUUUUUUCUAAAUG;
(SEQ ID NO: 7)
GAUCCAACUUUUUUCUAA;
(SEQ ID NO: 8)
CAUAGGGAUCCAACUUUUUUC;
or
(SEQ ID NO: 9)
CAUAGGGAUCCAACUUUUU.

In some embodiments, the ASO comprises a uracil complementary to an adenine located at position 429 of the SEQ ID NO: 16, position 229 of the SEQ ID NO: 17, or position 129 of the SEQ ID NO: 18.

In some embodiments, the ASO comprises 3 to 16 nucleotides upstream to the uracil.

In some embodiments, the ASO comprises: GCUUUCCUUCACUGUUGC (SEQ ID NO: 19); CUUUCCUUCACUGUUGCA (SEQ ID NO: 20); CUUUCCUUCACUGUUGCAAA (SEQ ID NO: 21); GGCUUUCCUUCACUGUUG (SEQ ID NO: 22); AAGGCUUUCCUUCACUGU (SEQ ID NO: 23); CCAAAGGCUUUCCUUCACUG (SEQ ID NO: 24); CAAAGGCUUUCCUUCACU (SEQ ID NO: 25); or UCCUUCACUGUUGCAAAGU (SEQ ID NO: 26).

In some embodiments, the subject comprises at least one mutation selected from the group consisting of: N1303K, W1282X, 4006delA, 4010del4, 4015delA, 4016insT, G1298A, T1299I, 4040delA, 4041 4046del6insTGT, 4048insCC, Q1313X, CFTRdele21, G1244E, T1246I, 3876delA, 3878delG, S1251N, L1254X, S1255P, S1255X, 3905insT, D1270N, R1283M, and Q1291R, wherein said X denotes translation termination.

In some embodiments, the at least one mutation is N1303K, W1282X, or both.

In some embodiments, treating comprises improving at least one clinical parameter of CF selected from the group consisting of: lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, and the need for antibiotic therapy for sinopulmonary signs or symptoms.

In some embodiments, the ASO comprises a chemically modified backbone.

In some embodiments, the chemically modified backbone comprises: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the composition further comprises a pharmaceutically acceptable carrier.

In some embodiments, the composition is for use in inducing the skipping of exon 23 or exon 24 of the CFTR pre-mRNA.

In some embodiments, the composition is an inhalation composition.

In some embodiments, the composition is for use in the treatment of CF.

In some embodiments, the at least one ASO comprises a sequence selected from the group consisting of SEQ ID Nos.: 4-14, and 19-30.

In some embodiments, the CF drug is an antibiotic drug, a bronchodilator, a corticosteroid, or any combination thereof.

In some embodiments, the compound is an ASO.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B are graphs showing CFTR function in HEK 293 cells transiently transfected with CFTR del Ex24 measured using the membrane potential sensitive FLIPR® dye. Following 5 min baseline measurement, CFTR was activated by forskolin (FSK) (10 uM) and VX-770 (luM). CFTR inhibitor (CFTRinh-172, 10 uM) was then added to inactivate CFTR.

FIG. 2 is a micrograph of a gel electrophoresis showing that synthetic antisense oligonucleotides (ASO) induce skipping over exon 24 of the CFTR pre-mRNA.

FIGS. 3A-3B are a micrograph of gel electrophoresis (3A) and a graph (3B) showing that synthetic antisense oligonucleotides (ASO) induce skipping over exon 23 of the CFTR pre-mRNA.

FIGS. 4A-4B are a micrograph and a vertical bar graph. (4A) gel electrophoresis analysis showing that the ASO effect (e.g., exon 23 skipping) is highly significant under nonsense-mediated decay (NMD) inhibition with SMG1 inhibitor. (4B) a graph showing that incubation of cells in the presence of 0.3 μg of SMG1, a NMD-inhibitor, increased mRNA levels.

FIG. 5 is micrographs of western blot analyses using an anti-CFTR antibody (upper panel) or an anti-Calnexin antibody (as control; lower panel). In 16HBEge W1282X CFTR proteins are not detectable, whereas skipping over exon 23 lead to the production of a mature (and deleted) CFTR protein.

### DETAILED DESCRIPTION

### Method of Treatment

According to some embodiments, a method for treating cystic fibrosis (CF) in a subject is provided. In some embodiments, the method comprises administering to the subject a therapeutically effective amount of a splicing modulator, wherein the splicing modulator induces the skipping of exon 23, exon 24, or both, of the cystic fibrosis transmembrane conductance regulator (CFTR) pre-mRNA, thereby treating CF in the subject.

In some embodiments, the method further comprises administering to the subject a therapeutically effective amount of one or more CFTR modifiers.

In some embodiments, the CFTR modifier increases the duration of the CFTR gate being open, chloride flow through the CFTR gate, CFTR protein proper folding, the number of CFTR anchored to the cell membrane, or any combination thereof. Each possibility represents a separate embodiment of the invention.

In some embodiment, the modifier is selected from: potentiator, corrector, and amplifier.

As used herein, the term “potentiator” refers to any agent that increases the probability that a defective CFTR will be open and therefore allows chloride ions to pass through the channel pore.

As used herein, the term “corrector” refers to any agent that assists in proper CFTR channel folding so as to enable its trafficking to the cell membrane.

As used herein, the term “amplifier” refers to any agent that induces a cell to increase its CFTR protein production rates or yields, therefore resulting in an increased amount of the CFTR protein.

In some embodiments, the modifier is selected from ivacaftor, lumacaftor, tezacaftor, VX-659, VX-445, VX-152, or VX-440.

In some embodiments, the modifier is ivacaftor, lumacaftor, tezacaftor, VX-659, VX-445, VX-152, or VX-440, or any combination thereof.

### Antisense Oligonucleotides

In some embodiments, the method comprises administering a splicing modulator which is at least one synthetic antisense oligonucleotide (ASO).

In some embodiments, the ASO is chemically modified. In some embodiments, the chemical modification is a modification of a backbone of the ASO. In some embodiments, the chemical modification is a modification of a sugar of the ASO. In some embodiments, the chemical modification is a modification of a nucleobase of the ASO. In some embodiments, the chemical modification increases stability of the ASO in a cell. In some embodiments, the chemical modification increases stability of the ASO in vivo. In some embodiments, the chemical modification increases the ASO's ability to modulate splicing. In some embodiments, the chemical modification increases the ASO's ability to induce skipping of exon 23, exon 24, or both. In some embodiments, the chemical modification increases the half-life of the ASO. In some embodiments, the chemical modification inhibits polymerase extension from the 3′ end of the ASO. In some embodiments, the chemical modification inhibits recognition of the ASO by a polymerase. In some embodiments, the chemical modification inhibits double-strand trigged degradation. In some embodiments, the chemically modified ASO does not trigger nucleic acid double-stranded degradation upon binding a CFTR pre-mRNA. In some embodiments, the chemical modification inhibits RISC-mediated degradation. In some embodiments, the chemical modification inhibits RISC-mediated degradation or any parallel nucleic acid degradation pathway.

In some embodiments, the ASO is devoid of a labeling moiety. In some embodiments, the ASO is not labeled. In some embodiments, the ASO does not emit a detectable signal or does not comprise moieties capable of being recognized so as to enable nucleic acid detection (e.g., digoxigenin and fluorescently labeled anti-DIG antibody). In some embodiments, a detectable signal comprises a dye or an emitting energy which provides detection of a compound, e.g., a polynucleotide, in vivo or in vitro. In some embodiments, a detectable signal comprises: a fluorescent signal, a chromatic signal, or a radioactive signal.

In some embodiments, the ASO is devoid of radioactive nucleobase(s);
digoxigenin, streptavidin, biotin, a fluorophore, hapten label, CLICK label, amine label, or thiol label.

In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the ASO comprises at least 14 bases, at least 15 bases, at least 16 bases, at least 17 bases, at least 18 bases, at least 19 bases, at least 20 bases, at least 21 bases, at least 22 bases, at least 23 bases, at least 24 bases, or at least 25 bases, or any value and range therebetween. Each possibility represents a separate embodiment of the invention.

In some embodiments, the ASO comprises 14 to 25 bases, 14 to 24 bases, 14 to 23 bases, 14 to 22 bases, 14 to 21 bases, 14 to 20 bases, 14 to 19 bases, or 14 to 18 bases, or 14 to 17 bases. Each possibility represents a separate embodiment of the invention. in some embodiments, the ASO comprises 17 to 22 bases.

In some embodiments, the ASO is complementary to a sequence comprising or consisting of:

(SEQ ID NO: 15)
UAAAAUUUUUUAAUAUUCUACAAUUAACAAUUAUCUCAAUUUCUUUAU
UCUAAAGACAUUGGGAUUAGAAAAAUGUUCACAAGGGACUCCAAAUAU
UGCUGUAGUAUUUGUUUCUUAAAAGAAUGAUACAAAGCAGACAUGAUA
AAAUAUUAAAAUUUGAGAGAACUUGAUGGUAAGUACAUGGGUGUUUCU
UAUUUUAAAAUAAUUUUUCUACUUGAAAUAUUUUACAAUACAAUAAGG
GAAAAAUAAAAAGUUAUUUAAGUUAUUCAUACUUUCUUCUUCUUUUCU
UUUUUGCUAUAGAAAGUAUUUAUUUUUUCUGGAACAUUUAGAAAAAAC
UUGGAUCCCUAUGAACAGUGGAGUGAUCAAGAAAUAUGGAAAGUUGCA
GAUGAGGUAAGGCUGCUAACUGAAAUGAUUUUGAAAGGGGUAACUCAU
ACCAACACAAAUGGCUGAUAUAGCUGACAUCAUUCUACACACUUUGUG
UGCAUGUAUGUGUGUGCACAACUUUAAAAUGGAGUACCCUAACAUACC
UGGAGCAACAGGUACUUUUGACUGGACCUACCCCUAACUGAAAUGAUU
UUGAAAGAGGUACUCAUACCAACACAAAUGGUUGAUAUGGCUAAGAUC
AUUCUACACACUUUGUGUGCAUGUAUUUCUGUGCACAACUUCAAAAUG
GAGUACCCUAAAAUACC.

In some embodiments, the ASO is complementary to a sequence comprising or consisting of:

(SEQ ID NO: 1)
UAAAAUUUUUUAAUAUUCUACAAUUAACAAUUAUCUCAAUUUCUUUAU
UCUAAAGACAUUGGGAUUAGAAAAAUGUUCACAAGGGACUCCAAAUAU
UGCUGUAGUAUUUGUUUCUUAAAAGAAUGAUACAAAGCAGACAUGAUA
AAAUAUUAAAAUUUGAGAGAACUUGAUGGUAAGUACAUGGGUGUUUCU
UAUUUUAAAAUAAUUUUUCUACUUGAAAUAUUUUACAAUACAAUAAGG
GAAAAAUAAAAAGUUAUUUAAGUUAUUCAUACUUUCUUCUUCUUUUCU
UUUUUGCUAUAGAAAGUAUUUAUUUUUUCUGGAACAUUUAGAAAAAAG
UUGGAUCCCUAUGAACAGUGGAGUGAUCAAGAAAUAUGGAAAGUUGCA
GAUGAGGUAAGGCUGCUAACUGAAAUGAUUUUGAAAGGGGUAACUCAU
ACCAACACAAAUGGCUGAUAUAGCUGACAUCAUUCUACACACUUUGUG
UGCAUGUAUGUGUGUGCACAACUUUAAAAUGGAGUACCCUAACAUACC
UGGAGCAACAGGUACUUUUGACUGGACCUACCCCUAACUGAAAUGAUU
UUGAAAGAGGUACUCAUACCAACACAAAUGGUUGAUAUGGCUAAGAUC
AUUCUACACACUUUGUGUGCAUGUAUUUCUGUGCACAACUUCAAAAUG
GAGUACCCUAAAAUACC.

In some embodiments, the ASO is complementary to a sequence comprising or consisting of:

(SEQ ID NO: 2)
AAAUAAUUUUUCUACUUGAAAUAUUUUACAAUACAAUAAGGGAAAAAU
AAAAAGUUAUUUAAGUUAUUCAUACUUUCUUCUUCUUUUCUUUUUUGC
UAUAGAAAGUAUUUAUUUUUUCUGGAACAUUUAGAAAAAAGUUGGAUC
CCUAUGAACAGUGGAGUGAUCAAGAAAUAUGGAAAGUUGCAGAUGAGG
UAAGGCUGCUAACUGAAAUGAUUUUGAAAGGGGUAACUCAUACCAACA
CAAAUGGCUGAUAUAGCUGACAUCAUUCUACACACUUUGUGUGCAUGU
AUG.

In some embodiments, the ASO has at least 75%, at least 80%, at least 85%, at least 90%, at least 95%, at least 99%, or 100% complementarity to any one of SEQ ID NO: 1, SEQ ID NO: 15, and SEQ ID NO: 2, or any value and range therebetween. Each possibility represents a separate embodiment of the invention. In some embodiments, the ASO has 70-80%, 75-85%, 80-90%, 85-95%, 90-99%, or 95-100% complementarity to any one of SEQ ID NO: 1, SEQ ID NO: 15, and SEQ ID NO: 2. Each possibility represents a separate embodiment of the invention.

In some embodiments, the ASO is complementary to a sequence comprising or consisting of:

(SEQ ID NO: 31)
CAAAAUGGGCAUUUUCAAUCUUUUUGUCAUUAGUAAAGGUCAGUGAUA
AAGGAAGUCUGCAUCAGGGGUCCAAUUCCUUAUGGCCAGUUUCUCUAU
UCUGUUCCAAGGUUGUUUGUCUCCAUAUAUCAACAUUGGUCAGGAUUG
AAAGUGUGCAACAAGGUUUGAAUGAAUAAGUGAAAAUCUUCCACUGGU
GACAGGAUAAAAUAUUCCAAUGGUUUUUAUUGAAGUACAAUACUGAAU
UAUGUUUAUGGCAUGGUACCUAUAUGUCACAGAAGUGAUCCCAUCACU
UUUACCUUAUAGGUGGGCCUCUUGGGAAGAACUGGAUCAGGGAAGAGU
ACUUUGUUAUCAGCUUUUUUGAGACUACUGAACACUGAAGGAGAAAUC
CAGAUCGAUGGUGUGUCUUGGGAUUCAAUAACUUUGCAACAGUGUAGG
AAAGCCUUUGGAGUGAUACCACAGGUGAGCAAAAGGACUUAGCCAGAA
AAAAGGCAACUAAAUUAUAUUUUUUACUGCUAUUUGAUACUUGUACUC
AAGAAAUUCAUAUUACUCUGCAAAAUAUAUUUGUUAUGCAUUGCUGUC
UUUUUUCUCCAGUGCAGUUUUCUCAUAGGCAGAAAAGAUGUCUCUAAA
AGUUUGGAAUUCUCAAAUUCUGGUUAUUGAAAUGUUCAUAGCUUUGAU
AGUGUUUUUCAGAAGACCAAAUUUACAGUGGGAGCCUUGGGCUUUUGU
UUUUUAACAGCUCUUUUUUGUUCCUGCUUCAGUGGC.

In some embodiments, the ASO is complementary to a sequence comprising or consisting of:

(SEQ ID NO: 16)
CAAAAUGGGCAUUUUCAAUCUUUUUGUCAUUAGUAAAGGUCAGUGAUA
AAGGAAGUCUGCAUCAGGGGUCCAAUUCCUUAUGGCCAGUUUCUCUAU
UCUGUUCCAAGGUUGUUUGUCUCCAUAUAUCAACAUUGGUCAGGAUUG
AAAGUGUGCAACAAGGUUUGAAUGAAUAAGUGAAAAUCUUCCACUGGU
GACAGGAUAAAAUAUUCCAAUGGUUUUUAUUGAAGUACAAUACUGAAU
UAUGUUUAUGGCAUGGUACCUAUAUGUCACAGAAGUGAUCCCAUCACU
UUUACCUUAUAGGUGGGCCUCUUGGGAAGAACUGGAUCAGGGAAGAGU
ACUUUGUUAUCAGCUUUUUUGAGACUACUGAACACUGAAGGAGAAAUC
CAGAUCGAUGGUGUGUCUUGGGAUUCAAUAACUUUGCAACAGUGAAGG
AAAGCCUUUGGAGUGAUACCACAGGUGAGCAAAAGGACUUAGCCAGAA
AAAAGGCAACUAAAUUAUAUUUUUUACUGCUAUUUGAUACUUGUACUC
AAGAAAUUCAUAUUACUCUGCAAAAUAUAUUUGUUAUGCAUUGCUGUC
UUUUUUCUCCAGUGCAGUUUUCUCAUAGGCAGAAAAGAUGUCUCUAAA
AGUUUGGAAUUCUCAAAUUCUGGUUAUUGAAAUGUUCAUAGCUUUGAU
AGUGUUUUUCAGAAGACCAAAUUUACAGUGGGAGCCUUGGGCUUUUGU
UUUUUAACAGCUCUUUUUUGUUCCUGCUUCAGUGGC.

In some embodiments, the ASO is complementary to a sequence comprising or consisting of:

(SEQ ID NO: 17)
AAAAUAUUCCAAUGGUUUUUAUUGAAGUACAAUACUGAAUUAUGUUUA
UGGCAUGGUACCUAUAUGUCACAGAAGUGAUCCCAUCACUUUUACCUU
AUAGGUGGGCCUCUUGGGAAGAACUGGAUCAGGGAAGAGUACUUUGUU
AUCAGCUUUUUUGAGACUACUGAACACUGAAGGAGAAAUCCAGAUCGA
UGGUGUGUCUUGGGAUUCAAUAACUUUGCAACAGUGGAGGAAAGCCUU
UGGAGUGAUACCACAGGUGAGCAAAAGGACUUAGCCAGAAAAAAGGCA
ACUAAAUUAUAUUUUUUACUGCUAUUUGAUACUUGUACUCAAGAAAUU
CAUAUUACUCUGCAAAAUAU.

In some embodiments, the ASO has at least 75%, at least 80%, at least 85%, at least 90%, at least 95%, at least 99%, or 100% complementarity to any one of SEQ ID NO: 16, SEQ ID NO: 31, and SEQ ID NO: 17, or any value and range therebetween. Each possibility represents a separate embodiment of the invention. In some embodiments, the ASO has 70-80%, 75-85%, 80-90%, 85-95%, 90-99%, or 95-100% complementarity to any one of SEQ ID NO: 16, SEQ ID NO: 31, and SEQ ID NO: 17. Each possibility represents a separate embodiment of the invention.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

In some embodiments, the ASO comprises a mismatched base compared to any one of SEQ ID NO: 1, SEQ ID NO: 15, and SEQ ID NO: 2. In some embodiments, the ASO comprises at least one, at least two, or at least 3 mismatched bases compared to any one of SEQ ID NO: 1, SEQ ID NO: 15, and SEQ ID NO: 2, or any value and range therebetween. Each possibility represents a separate embodiment of the invention. In some embodiments, the ASO comprises one to two, one to three, two to three mismatched bases compared to any one of SEQ ID NO: 1, SEQ ID NO: 15, and SEQ ID NO: 2. Each possibility represents a separate embodiment of the invention.

In some embodiments, the ASO comprises at most one, at most two, at least three, at most four, or at most five mismatched bases compared to any one of SEQ ID NO: 1, SEQ ID NO: 15, and SEQ ID NO: 2, or any value and range therebetween. Each possibility represents a separate embodiment of the invention. In some embodiments, the ASO comprises one to two, one to three, one to four, one to five, two to three, two to four, two to five, three to four, three to five, or four to five mismatched bases compared to any one of SEQ ID NO: 1, SEQ ID NO: 15, and SEQ ID NO: 2. Each possibility represents a separate embodiment of the invention.

In some embodiments, the ASO comprises a mismatched base compared to any one of SEQ ID NO: 16, SEQ ID NO: 31, and SEQ ID NO: 17. In some embodiments, the ASO comprises at least one, at least two, or at least 3 mismatched bases compared to any one of SEQ ID NO: 16, SEQ ID NO: 31, and SEQ ID NO: 17, or any value and range therebetween. Each possibility represents a separate embodiment of the invention. In some embodiments, the ASO comprises one to two, one to three, two to three mismatched bases compared to any one of SEQ ID NO: 16, SEQ ID NO: 31, and SEQ ID NO: 17. Each possibility represents a separate embodiment of the invention.

In some embodiments, the ASO comprises at most one, at most two, at least three, at most four, or at most five mismatched bases compared to any one of SEQ ID NO: 16, SEQ ID NO: 31, and SEQ ID NO: 17, or any value and range therebetween. Each possibility represents a separate embodiment of the invention. In some embodiments, the ASO comprises one to two, one to three, one to four, one to five, two to three, two to four, two to five, three to four, three to five, or four to five mismatched bases compared to any one of SEQ ID NO: 16, SEQ ID NO: 31, and SEQ ID NO: 17. Each possibility represents a separate embodiment of the invention.

In some embodiments, the ASO comprises one mismatched base at most, wherein the mismatched base is located not more than 1, 2, 3, or 4 bases from the 5′ prime end of the ASO, or any value and range therebetween. Each possibility represents a separate embodiment of the invention. In some embodiments, the ASO comprises one mismatched base at most, wherein the mismatched base is located not more than 1, 2, or 3 bases from the 5′ prime end of the ASO, or any value and range therebetween. Each possibility represents a separate embodiment of the invention. In some embodiments, the ASO comprises one mismatched base at most, wherein the mismatched base is located not more than 1, or 2 bases from the 5′ prime end of the ASO, or any value and range therebetween. Each possibility represents a separate embodiment of the invention.

In some embodiments, the ASO comprises one mismatched base at most, wherein the mismatched base is located not more than 1, 2, 3, or 4 bases from 3 prime end of the ASO, or any value and range therebetween. Each possibility represents a separate embodiment of the invention. In some embodiments, the ASO comprises one mismatched base at most, wherein the mismatched base is located not more than 1, 2, or 3 bases from 3 prime end of the ASO, or any value and range therebetween. Each possibility represents a separate embodiment of the invention. In some embodiments, the ASO comprises one mismatched base at most, wherein the mismatched base is located not more than 1, or 2 bases from 3 prime end of the ASO, or any value and range therebetween. Each possibility represents a separate embodiment of the invention.

In some embodiments, the ASO is complementary to a sequence comprising or consisting of:

(SEQ ID NO: 3)
AAAGUAUUUAUUUUUUCUGGAACAUUUAGAAAAAAGUUGGAUCCCUAU
GAACAGUGGAGUGAUCAAGAAAUAUGGAAAGUUGCAGAUGAG.

In some embodiments, the ASO is complementary to a sequence comprising or consisting of:

(SEQ ID NO: 18)
GUGGGCCUCUUGGGAAGAACUGGAUCAGGGAAGAGUACUUUGUUAUCA
GCUUUUUUGAGACUACUGAACACUGAAGGAGAAAUCCAGAUCGAUGGU
GUGUCUUGGGAUUCAAUAACUUUGCAACAGUGAAGGAAAGCCUUUGGA
GUGAUACCACAG.

In some embodiments, the ASO has at least 75%, at least 80%, at least 85%, at least 90%, at least 95%, at least 99%, or 100% complementarity to SEQ ID NO: 3 or SEQ ID NO: 18, or any value and range therebetween. Each possibility represents a separate embodiment of the invention. In some embodiments, the ASO has 70-80%, 75-85%, 80-90%, 85-95%, 90-99%, or 95-100% complementarity to SEQ ID NO: 3 or SEQ ID NO: 18. Each possibility represents a separate embodiment of the invention.

In some embodiments, the ASO comprises a cytosine complementary to a guanine located at position 336 of SEQ ID NO: 1.

In some embodiments, the ASO comprises a cytosine complementary to a guanine located at position 136 of SEQ ID NO: 2.

In some embodiments, the ASO comprises a cytosine complementary to a guanine located at position 36 of SEQ ID NO: 3.

In some embodiments, the ASO comprises a uracil complementary to an adenine located at position 429 of SEQ ID NO: 16.

In some embodiments, the ASO comprises a uracil complementary to an adenine located at position 229 of SEQ ID NO: 17.

In some embodiments, the ASO comprises a uracil complementary to an adenine located at position 129 of said SEQ ID NO: 18.

In some embodiments, the ASO comprises at least 4 bases, at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, or at least 18 bases upstream to the cytosine complementary to the guanine located at position 336 of SEQ ID NO: 1, position 136 of SEQ ID NO: 2, or position 36 of SEQ ID NO: 3, or any value and range therebetween. In some embodiments, the ASO comprises 4 to 18 bases, 4 to 16 bases, 4 to 15 bases, 5 to 17 bases, 5 to 13 bases, 8 to 18 bases, 7 to 13 bases, 9 to 13 bases, 6 to 12 bases 10 to 14 bases, or 12 to 18 bases upstream to the cytosine complementary to the guanine located at position 336 of SEQ ID NO: 1, position 136 of SEQ ID NO: 2, or position 36 of SEQ ID NO: 3. Each possibility represents a separate embodiment of the invention.

In some embodiments, the ASO comprises at least 4 bases, at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, or at least 18 bases upstream to the uracil complementary to the adenine located at position 429 of SEQ ID NO: 16, position 229 of SEQ ID NO: 17, or position 129 of said SEQ ID NO: 18, or any value and range therebetween. In some embodiments, the ASO comprises 4 to 18 bases, 4 to 16 bases, 4 to 15 bases, 5 to 17 bases, 5 to 13 bases, 8 to 18 bases, 7 to 13 bases, 9 to 13 bases, 6 to 12 bases 10 to 14 bases, or 12 to 18 bases upstream to the uracil complementary to the adenine located at position 429 of SEQ ID NO: 16, position 229 of SEQ ID NO: 17, or position 129 of said SEQ ID NO: 18. Each possibility represents a separate embodiment of the invention.

In some embodiments, the ASO comprises:

(SEQ ID NO: 4)
CCAACUUUUUUCUAAAUGUUCC;
(SEQ ID NO: 5)
UCCAACUUUUUUCUAAAUGU;
(SEQ ID NO: 6)
GGAUCCAACUUUUUUCUAAAUG;
(SEQ ID NO: 7)
GAUCCAACUUUUUUCUAA;
(SEQ ID NO: 8)
CAUAGGGAUCCAACUUUUUUC;
or
(SEQ ID NO: 9)
CAUAGGGAUCCAACUUUUU.

In some embodiments, the ASO comprises: GCUUUCCUUCACUGUUGC (SEQ ID NO: 19); CUUUCCUUCACUGUUGCA (SEQ ID NO: 20); CUUUCCUUCACUGUUGCAAA (SEQ ID NO: 21); GGCUUUCCUUCACUGUUG (SEQ ID NO: 22); AAGGCUUUCCUUCACUGU (SEQ ID NO: 23); CCAAAGGCUUUCCUUCACUG (SEQ ID NO: 24); CAAAGGCUUUCCUUCACU (SEQ ID NO: 25); or UCCUUCACUGUUGCAAAGU (SEQ ID NO: 26).

In some embodiments, the ASO is complementary to the CFTR pre-mRNA (Accession number NM_000492). In some embodiments, the pre-mRNA is a wild type pre-mRNA. In some embodiments, the pre-mRNA is a mutated pre-mRNA. In some embodiments, the CFTR pre-mRNA comprises any one of: SEQ ID NO: 1, SEQ ID NO: 2, SEQ ID NO: 3, SEQ ID NO: 15, SEQ ID NO: 16, SEQ ID NO: 17, SEQ ID NO: 18, and SEQ ID NO: 31. In some embodiments, the ASO is complementary to comprises any one of: SEQ ID NO: 1, SEQ ID NO: 2, SEQ ID NO: 3, SEQ ID NO: 15. SEQ ID NO: 16, SEQ ID NO: 17, SEQ ID NO: 18, and SEQ ID NO: 31.

In some embodiments, the ASO comprises an active fragment of any one of SEQ ID Nos: 4-30.

As used herein, the term “active fragment” refers to a fragment that is 100% identical to a contiguous portion of the full nucleotide sequence of the ASO, providing that at least: 30%, 40%, 50%, 60%, 70%, 80% or 90% of the activity of the original ASO nucleotide sequence is retained, or any value and range therebetween. Each possibility represents a separate embodiment of the present invention.

In some embodiments, the ASO is specific to a CFTR pre-mRNA. As used herein, the term “specific” refers to both base pair specificity and also gene specificity. In some embodiments, the ASO is specific to the CFTR gene. In some embodiments, the ASO is specific to a splice silencing motif in CFTR. In some embodiments, the ASO is specific to a splice silencing sequence is CFTR. In some embodiments, the ASO is specific to a splice silencing region of CFTR. In some embodiments, the splice silencing is splice silencing of exon 23 or exon 24 of CFTR.

In some embodiments, the ASO binds the CFTR pre-mRNA with perfect complementarity. In some embodiments, the ASO does not bind any gene other than CFTR with perfect complementarity. In some embodiments, the ASO does not bind any gene other than CFTR with a complementarity of greater than 70, 75, 80, 85, 90, 95, 97, 99 or 100%. Each possibility represents a separate embodiment of the invention. In some embodiments, the ASO does not bind any gene other than CFTR with a complementarity of greater than 90%. In some embodiments, the ASO binds any one of: SEQ ID NO: 1, SEQ ID NO: 2, SEQ ID NO: 3, SEQ ID NO: 15, SEQ ID NO: 16, SEQ ID NO: 17, SEQ ID NO: 18, and SEQ ID NO: 31 with perfect complementarity. In some embodiments, the ASO does not bind any sequence other than SEQ ID NO: 1, SEQ ID NO: 2, SEQ ID NO: 3, SEQ ID NO: 15, SEQ ID NO: 16, SEQ ID NO: 17, SEQ ID NO: 18, or SEQ ID NO: 31 with perfect complementarity. In some embodiments, the ASO does not bind any sequence other than SEQ ID NO: 1, SEQ ID NO: 2, SEQ ID NO: 3, SEQ ID NO: 15, SEQ ID NO: 16, SEQ ID NO: 17, SEQ ID NO: 18, or SEQ ID NO: 31 with complementarity of greater than 70, 75, 80, 85, 90, 95, 97, 99 or 100%. Each possibility represents a separate embodiment of the invention. In some embodiments, the ASO does not bind any sequence other than SEQ ID NO: 1, SEQ ID NO: 2, SEQ ID NO: 3, SEQ ID NO: 15, SEQ ID NO: 16, SEQ ID NO: 17, SEQ ID NO: 18, or SEQ ID NO: 31 with a complementarity of greater than 90%. In some embodiments, the ASO does not bind with perfect complementarity to anywhere in the genome of a cell other than within CFTR. In some embodiments, the ASO does not bind with complementarity of greater than 70, 75, 80, 85, 90, 95, 97, 99 or 100% to anywhere in the genome of a cell other than within CFTR. Each possibility represents a separate embodiment of the invention. In some embodiments, the cell is a mammalian cell. In some embodiments, the mammal is a human.

In some embodiments, the ASO modulates expression of CFTR. In some embodiments, the ASO modulates splicing of CFTR. In some embodiments, the ASO modulates splicing of exon 23, exon 24, or both of CFTR. In some embodiments, the ASO does not cause an off-target effect. In some embodiments, off-target is a target other than CFTR. In some embodiments, off-target is a target other than splicing of exon 23, exon 24, or both of CFTR. In some embodiments, the ASO does not substantially or significantly modulate expression of a gene other than CFTR. In some embodiments, the ASO does not substantially or significantly modulate splicing of a gene other than CFTR. In some embodiments, the ASO does not substantially or significantly modulate splicing of an exon other than exon 23, exon 24, or both of CFTR. In some embodiments, substantial modulation of expression is a change in expression of at least 5, 10, 15, 20, 25, 30, 35, 40, 45 or 50%. Each possibility represents a separate embodiment of the invention. In some embodiments, substantial modulation of expression is a change in expression of at least 20%.

In some embodiments, the ASO is complementary to an exon-intron junction. In some embodiments the exon is exon 23 or exon 24 of the CFTR pre-mRNA. As used herein, an exon-intron junction comprising a portion of or all of exon 23 or exon 24 may be referred to as exon 23-intron junction or exon 24-intron junction. In some embodiments, an exon 23-intron junction or exon 24-intron junction comprises the 5′ prime end of exon 23 or exon 24. In some embodiments, an exon 23-intron junction or exon 24-intron junction comprises the 3′ prime end of exon 23 or exon 24. In some embodiments, an exon 23-intron junction or exon 24-intron junction comprises the complete sequence of exon 23 or exon 24. In some embodiments, any one of SEQ ID NO: 1, SEQ ID NO: 15, and SEQ ID NO: 2, comprises or consists of an exon 24-intron junction. In some embodiments, any one of SEQ ID NO: 16, SEQ ID NO: 31, and SEQ ID NO: 17, comprises or consists of an exon 23-intron junction.

In some embodiments, the ASO is at least 70%, at least 80%, at least 85%, at least 90%, at least 95%, at least 99%, or 100% complementary to an exon 24-intron junction of the CFTR pre-mRNA, or any value and range therebetween. Each possibility represents a separate embodiment of the invention. In some embodiments, the ASO is 70-85%, 80-90%, 85-95%, 90-99%, or 95-100% complementary to an exon 23-intron junction or an exon 24-intron junction of the CFTR pre-mRNA. Each possibility represents a separate embodiment of the invention.

In some embodiments, the ASO is at least 70%, at least 80%, at least 85%, at least 90%, at least 95%, at least 99%, or 100% complementary to a sequence located at positions 282-318 of any one of SEQ ID NO: 1 and SEQ ID NO: 15, or positions 182-118 of SEQ ID NO: 2, or any value and range therebetween. Each possibility represents a separate embodiment of the invention. In some embodiments, the ASO is 70-85%, 80-90%, 85-99%, or 95-100% complementary to a sequence located at positions 282-318 of any one of SEQ ID NO: 1 and SEQ ID NO: 15, or positions 82-118 of SEQ ID NO: 2. Each possibility represents a separate embodiment of the invention.

In some embodiments, the ASO is at least 70%, at least 80%, at least 85%, at least 90%, at least 95%, at least 99%, or 100% complementary to a sequence located at positions 275-325 of any one of SEQ ID NO: 16 and SEQ ID NO: 31, or positions 75-125 of SEQ ID NO: 17, or any value and range therebetween. Each possibility represents a separate embodiment of the invention. In some embodiments, the ASO is 70-85%, 80-90%, 85-99%, or 95-100% complementary to a sequence located at positions 275-325 of any one of SEQ ID NO: 16 and SEQ ID NO: 31, or positions 75-125 of SEQ ID NO: 17. Each possibility represents a separate embodiment of the invention.

In some embodiments, the ASO is at least at least 70%, at least 80%, at least 85%, at least 90%, at least 95%, at least 99%, or 100% complementary to a sequence located at positions 376-421 of any one of SEQ ID NO: 1 and SEQ ID NO: 15, or positions 176-221 of SEQ ID NO: 2, or any value and range therebetween. Each possibility represents a separate embodiment of the invention. In some embodiments, the ASO is 70-85%, 80-90%, 85-99%, or 95-100% complementary to a sequence located at positions 376-421 of any one of SEQ ID NO: 1 and SEQ ID NO: 15, or positions 176-221 of SEQ ID NO: 2. Each possibility represents a separate embodiment of the invention.

In some embodiments, the ASO is at least at least 70%, at least 80%, at least 85%, at least 90%, at least 95%, at least 99%, or 100% complementary to a sequence located at positions 435-485 of any one of SEQ ID NO: 16 and SEQ ID NO: 31, or positions 235-285 of SEQ ID NO: 17, or any value and range therebetween. Each possibility represents a separate embodiment of the invention. In some embodiments, the ASO is 70-85%, 80-90%, 85-99%, or 95-100% complementary to a sequence located at positions 435-485 of any one of SEQ ID NO: 16 and SEQ ID NO: 31, or positions 235-285 of SEQ ID NO: 17. Each possibility represents a separate embodiment of the invention.

In some embodiments, an ASO complementary to a sequence located at positions 282-318 of any one of SEQ ID NO: 1 and SEQ ID NO: 15, or positions 82-118 of SEQ ID NO: 2 comprises or consists of any one of: AAAUAAAUACUUUCUAUAGC (SEQ ID NO: 10); AAUACUUUCUAUAGCAAAAA (SEQ ID NO: 11), or AUACUUUCUAUAGCAAAAAAG (SEQ ID NO: 12).

In some embodiments, an ASO complementary to a sequence located at positions 376-421 of any one of SEQ ID NO: 1 and SEQ ID NO: 15, or positions 176-221 of SEQ ID NO: 2 comprises or consists of CAGCCUUACCUCAUCUGCA (SEQ ID NO: 13), or CAGUUAGCAGCCUUACCUC (SEQ ID NO: 14).

In some embodiments, an ASO complementary to a sequence located at positions 275-325 of any one of SEQ ID NO: 16 and SEQ ID NO: 31, or positions 75-125 of SEQ ID NO: 17 comprises or consists of any one of: CAAGAGGCCCACCUAUAAG (SEQ ID NO: 27), or CCACCUAUAAGGUAAAAGUG (SEQ ID NO: 28).

In some embodiments, an ASO complementary to a sequence located at positions 435-485 of any one of SEQ ID NO: 16 and SEQ ID NO: 31, or positions 235-285 of SEQ ID NO: 17 comprises or consists of CCUUUUGCUCACCUGUGGU (SEQ ID NO: 29), or CUCACCUGUGGUAUCACU (SEQ ID NO: 30).

In some embodiments, an ASO as disclosed herein targets, complements, induces, or any combination thereof, the skipping of exon 23 or exon 24 of CFTR pre-mRNA transcribed from a mutated allele of the CFTR gene. In some embodiments, an ASO as disclosed herein does not target, complement, induce, or any combination thereof, the skipping of exon 23 or exon 24 of CFTR pre-mRNA transcribed from a wild type allele of the CFTR gene. In some an ASO as disclosed herein targets, complements, induces, or any combination thereof at least 2 fold more efficiently, at least 3 fold more efficiently, at least 5 fold more efficiently, at least 7 fold more efficiently, at least 10 fold more efficiently, at least 20 fold more efficiently, at least 50 fold more efficiently, or at least 100 fold more efficiently, the skipping of exon 23 or exon 24 of CFTR pre-mRNA transcribed from a mutated allele of the CFTR gene compared to the wild type allele of the CFTR gene, or any value and range therebetween. Each possibility represents a separate embodiment of the invention. In some an ASO as disclosed herein targets, complements, induces, or any combination thereof 2-10 fold more efficiently, 3-50 fold more efficiently, 5-100 fold more efficiently, 7-20 fold more efficiently, 2-40 fold more efficiently, 2-25 fold more efficiently, 50-150 fold more efficiently, or 2-100 fold more efficiently, the skipping of exon 23 or exon 24 of CFTR pre-mRNA transcribed from a mutated allele of the CFTR gene compared to the wild type allele of the CFTR gene. Each possibility represents a separate embodiment of the invention.

In some embodiments, an ASO of the invention fully complements with a mutated allele of the CFTR gene. As used herein, the term “fully complements” refers to 100% hybridization, meaning the mutated CFTR allele and the ASO represent a reversed and complementary nucleic acid sequence versions of one another, as would be apparent to one of ordinary skill in the art of molecular biology. In some embodiments, an ASO of the invention partially complements with the wild type allele of the CFTR gene. As used herein, the term “partially” refers to any value or range lower than 100%. In some embodiments, embodiments, the ASO of the invention and the wild type CFTR allele represent a reversed-and-complementary nucleic acid sequence version of one another which differ by at least one nucleotide, e.g., comprising at least one mismatched nucleotide.

In some embodiments, the ASO of the invention, and method of using same, provide the exclusion of a mutated: exon 23, exon 24, or both from the CFTR pre-mRNA, whereas the wild type: exon 23, exon 24, or both, is retained, remains included, is not being excluded, or any equivalent thereof, in a CFTR pre-mRNA.

In some embodiments, the ASO of the invention, and method of using same, provide the exclusion of only a mutated: exon 23, exon 24, or both, from a CFTR pre-mRNA, whereas the wild type, e.g., non-mutated: exon 23, exon 24, or both, is retained, remains included, is not being excluded, or any equivalent thereof, from the wild type CFTR pre-mRNA.

In some embodiments, the mutation is a CF-conferring mutation. As used herein, the term “CF-conferring mutation” refers to any mutation which induces, promotes, relates, or propagates the development of Cystic fibrosis disease or symptoms associated therewith in a subject harboring or comprising the mutation.

In some embodiments, the mutation is in exon 23, exon 24, or both, of the CFTR encoding gene.

In some embodiments, the subject comprises a mutation. In some embodiments, the subject comprises a missense mutation. In some embodiments, the subject comprises a nonsense mutation. In some embodiments, the subject comprises a substitution mutation in the CFTR encoding gene, pre-mRNA encoded therefrom, or protein product thereof. In some embodiments, the subject comprises one or more mutations selected from: N1303K, 4006delA, 4010del4, 4015delA, 4016insT, G1298A, T1299I, 4040delA, 4041 4046del6insTGT, 4048insCC, Q1313X, and CFTRdele21. In some embodiments, the subject comprises one or more mutations selected from: W1282X, G1244E, T1246I, 3876delA, 3878delG, S1251N, L1254X, S1255P, S1255X, 3905insT, D1270N, R1283M, Q1291R, wherein said X denotes translation termination. In some embodiments, the subject comprises a wild type (i.e., not mutated) exon 23 or exon 24. In some embodiments, the subject comprises at least one CF-inducing mutation residing in the CFTR gene, or mRNA transcribed therefrom, wherein the mutation does not reside in exon 23 or exon 24, affect exon 23 or exon 24 inclusion or exclusion from the mature mRNA, or both. In some embodiments, the subject comprises both a wild type exon 24, and at least one CF-inducing mutation residing in the CFTR gene, or mRNA transcribed therefrom, wherein the mutation does not reside in exon 24, affect exon 24 inclusion or exclusion from the mature mRNA, or both. In some embodiments, the subject comprises both a wild type exon 23, and at least one CF-inducing mutation residing in the CFTR gene, or mRNA transcribed therefrom, wherein the mutation does not reside in exon 23, affect exon 23 inclusion or exclusion from the mature mRNA, or both.

In some embodiments, the subject is homozygous to one or more of the aforementioned mutations. In some embodiments, the subject is heterozygous to one or more of the aforementioned mutations. In some embodiments, a subject treated according to the method of the invention, comprises or is characterized by having a mixture of a wild type full-length and fully functional CFTR protein encoded from the wild type allele and a deleterious CFTR protein encoded from the pre-mRNA from which exon 23 or exon 24 was excluded using the ASO of the invention. In some embodiments, the ASO of the invention does not reduce the level of the wild type full-length and fully functional CFTR protein in a subject, e.g., heterozygous to a mutation as disclosed hereinabove. In some embodiments, the subject is further heterozygous to additional one or more mutations, wherein the additional one or more mutations is located in the CFTR pre-mRNA in an exon other than exon 23 or/and exon 24. In some embodiments, the subject is homozygous or heterozygous to the one or more CF-conferring mutations disclosed herein, e.g., N1303K, W1282X, and is further heterozygous to an additional one or more mutations located in any exon of the CFTR pre-mRNA other than exon 23 or/and exon 24.

In some embodiments, “a mutation” as used herein, refers to any nucleotide substitution or modification which renders a partially or fully non-functional CFTR protein. In some embodiments, “a mutation” as used herein, refers to a nucleotide substitution or modification which induces or results in a “Cystic fibrosis phenotype” in a subject harboring or comprising the mutation.

In some embodiments, a modification comprises insertion, deletion, inversion, or a combination thereof, as long as the modification results in a Cystic fibrosis phenotype in a subject harboring or comprising the modification.

As used herein, the term “Cystic fibrosis phenotype” encompasses any symptom or manifestation related to Cystic fibrosis. Methods for diagnosing Cystic fibrosis and/or symptoms associated therewith are common and would be apparent to one of ordinary skill in the art.

In some embodiments, the subject comprises an Asparagine to Lysine substitution in the CFTR protein. In some embodiments, the subject comprises a substitution in position 1303 or the CFTR protein. In some embodiments, the subject comprises a N1303K substitution in the CFTR protein.

In some embodiments, the subject comprises a Tryptophan substituted with a translation termination codon in the CFTR protein. In some embodiments, the subject comprises a substitution in position 1282 of the CFTR protein. In some embodiments, the subject comprises a W1282X substitution in the CFTR protein, wherein the X denotes translation termination.

In some embodiments the subject is afflicted with Cystic fibrosis.

In some embodiments, the method is directed to improving at least one clinical parameter of CF in the subject, selected from: lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, or the need for antibiotic therapy for sinopulmonary signs or symptoms.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “condition” includes anatomic and physiological deviations from the normal that constitute an impairment of the normal state of the living animal or one of its parts, that interrupts or modifies the performance of the bodily functions.

As used herein, the terms “subject” or “individual” or “animal” or “patient” or “mammal,” refers to any subject, particularly a mammalian subject, for whom therapy is desired, for example, a human.

In some embodiments, there is provided a method for treating cystic fibrosis (CF) in a subject in need thereof, comprising administering to the subject a therapeutically effective amount of a synthetic antisense oligonucleotide (ASO), wherein the ASO induces the skipping of exon 24 of the cystic fibrosis transmembrane conductance regulator (CFTR) pre-mRNA, thereby treating CF in the subject, and wherein the ASO targets a CF-conferring mutation located in exon 24 of the CFTR pre-mRNA.

Composition

According to some embodiments, a composition comprising an ASO comprising 14 to 25 bases having at least 80% complementarity to a CFTR pre-mRNA, and characterized by inducing splicing activity of exon 23 or exon 24 of the CFTR pre-mRNA, is provided.

In some embodiments, the composition comprises a plurality of ASOs characterized by inducing splicing activity of different target pre-mRNA. In some embodiments, the composition comprises at least two ASOs as described herein, wherein the first ASO is characterized by inducing splicing activity of exon 23 of the CFTR pre-mRNA, and wherein the second ASO is characterized by inducing splicing activity of exon 243 or exon 24 of the CFTR pre-mRNA.

In some embodiments, the composition further comprises a pharmaceutically acceptable carrier.

The term “pharmaceutically acceptable carrier” as used herein refers to any of the standard pharmaceutical carriers known in the field such as sterile solutions, tablets, coated tablets, and capsules. Typically, such carriers contain excipients such as starch, milk, sugar, certain types of clay, gelatin, stearic acids or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, nano-particles, nano-emulsions, pastes, creams, salves, etc., and may be in unit-dosage forms suitable for administration of relatively precise dosages.

In some embodiments, the pharmaceutical composition is formulated for oral, administration. In some embodiments, the pharmaceutical composition is formulated for nasal administration. In some embodiments, the pharmaceutical composition is formulated for administration by inhalation. In some embodiments, the pharmaceutical composition is formulated for abdominal administration. In some embodiments, the pharmaceutical composition is formulated for subcutaneous administration. In some embodiments, the pharmaceutical composition is formulated for intra-peritoneal administration. In some embodiments, the pharmaceutical composition is formulated for intravenous administration.

In some embodiments, the pharmaceutical composition is formulated for systemic administration. In some embodiments, the pharmaceutical composition is formulated for administration to a subject. In some embodiments, the subject is a human subject. It will be understood by a skilled artisan that a pharmaceutical composition intended to administration to a subject should not have off-target effects, i.e. effects other than the intended therapeutic ones. In some embodiments, the pharmaceutical composition is devoid of a substantial effect on a gene other than CFTR. In some embodiments, the pharmaceutical composition is devoid of a substantial effect on splicing of an exon other than exon 23, exon 24, or both, of CFTR. In some embodiments, a substantial effect is one with a phenotypic result. In some embodiments, a substantial effect is a deleterious effect. In some embodiments, deleterious is with respect to the health and/or wellbeing of the subject.

In some embodiments, the composition administered by inhalation. In some embodiments, the composition is an inhalation composition. in some embodiments, the composition is a pharmaceutical composition.

Being a long-known and well-studied disease, certain drugs and agents are known in the art for the treatment of Cystic Fibrosis patients. Administrating a synthetic polynucleotide molecule according to the present invention with one or more of these drugs may be beneficial in achieving significant therapeutic results.

In some embodiments, the composition further comprises at least one additional anti-Cystic-Fibrosis agent (i.e., CF drug). In some embodiments, the additional anti-Cystic-Fibrosis agent is selected from: a CFTR-splicing-modulator (e.g., an ASO as disclosed and as described herein), Translational Read-Through agent, sodium epithelial channel (ENaC) inhibitor, a CFTR amplifier, a CFTR potentiator, or a CFTR corrector. In some embodiments, the CFTR-splicing-modulator has capability to induce or promote exon 24 exclusion from the mature CFTR mRNA; the Translational Read-Through agent is selected from 3-[5-(2-fluorophenyl)-1,2,4-oxadiazol-3-yl]benzoic acid (Ataluren), or ELX-02; the ENaC inhibitor is selected from: VX-371 (P-1037) or IONIS-ENAC-2.5Rx; the CFTR amplifier is PTI-428; the CFTR potentiator is selected from: N-(2,4-Di-tert-butyl-5-hydroxyphenyl)-4-oxo-1,4-dihydroquinoline-3-carboxamide (Ivacaftor), QBW251, PTI-808, or VX-561 (deuterated ivacaftor); the CFTR potentiator is N-(2,4-Di-tert-butyl-5-hydroxyphenyl)-4-oxo-1,4-dihydroquinoline-3-carboxamide (Ivacaftor); or the CFTR corrector is selected from: 3-{6-{[1-(2,2-difluoro-1,3-benzodioxol-5-yl)cyclopropanecarbonyl]amino}-3-methylpyridin-2-yl}benzoic acid (Lumacaftor), 1-(2,2-difluoro-1,3-benzodioxol-5-yl)-˜{N}-[1-[(2-{R})-2,3-dihydroxypropyl]-6-fluoro-2-(1-hydroxy-2-methylpropan-2-yl)indol-5-yl]cyclopropane-1-carboxamide (Tezacaftor), VX-659, VX-445, VX-152 and VX-440, GLPG2222, FDL169, or PTI-801.

In some embodiments, the pharmaceutical composition comprises the synthetic ASO of the invention. In some embodiments, the composition comprises at the ASO in an amount of at least 1 nM, at least 2.5 nM, at least 10 nM, or any value and range therebetween. Each possibility represents a separate embodiment of the invention. In some embodiments, the composition comprises at the ASO in an amount of 2.5 nM to 10 nM, 1 nM to 100 nM, 1 nM to 0.5 or 1 nM to 1 Each possibility represents a separate embodiment of the invention.

In some embodiments, an ASO as disclosed and as described hereinabove, or a pharmaceutical composition comprising thereof, is used in the modulation of splicing of a CFTR pre-mRNA transcribed from a CFTR gene having a mutated: exon 23, exon 24, or both.

The phrase “modulation of splicing” as used herein refers to affecting a change in the level of any RNA or mRNA variant produced by the CFTR native pre-mRNA. For example, modulation may mean e.g. causing an increase or decrease in the level of abnormal CFTR mRNA, causing an increase or decrease in the level of normal, full-length CFTR mRNA, causing an increase or decrease in the level of abnormal CFTR RNA or mRNA comprising a missense codon, and/or causing an increase or decrease in the level of abnormal CFTR RNA or mRNA comprising a premature termination codon (non-sense codon). It is therefore evident that any change in ratio between certain CFTR splicing variants is also considered to be the result of splicing modulation. Each possibility represents a separate embodiment of the invention. In certain embodiments, modulation means decreasing the level of abnormal CFTR mRNA. In some embodiments, the abnormal CFTR mRNA comprises a mutated: exon 23, exon 24, or both. In some embodiments, modulation means decreasing the level of an abnormal CFTR mRNA comprising a mutated: exon 23, exon 24, or both. In some embodiments, modulation means decreasing the level of an abnormal CFTR mRNA comprising a N1303K mutation, a W1282X mutation, or both.

In certain embodiments, the use is for reducing the level of an mRNA molecule comprising the mutated: exon 23, exon 24, or both. In some embodiments, the use is for reducing the level of an mRNA molecule comprising the nucleotide sequence set forth in SEQ ID NO: 1, SEQ ID NO: 2, SEQ ID NO: 3, SEQ ID NO: 16, SEQ ID NO: 17, or SEQ ID NO: 18. In some embodiments, the use is for increasing the level of CFTR mRNA lacking exon 23, exon 24, or both. In some embodiments, the use is for increasing the level of CFTR mRNA lacking SEQ ID NO: 3, SEQ ID NO: 18, or both. In some embodiments, the use is for correcting or improving chloride transport through the CFTR channel. In some embodiments, the use is for increasing the production of functional CFTR protein. In some embodiments, the use is for increasing the duration of the CFTR gate being open. In some embodiments, the use is for increasing the chloride flow through the CFTR gate. In some embodiments, the use is for increasing the CFTR protein proper folding. In some embodiments, the use is for increasing the number of CFTR anchored to the cell membrane.

In some embodiments, an ASO as disclosed and as described hereinabove, or a pharmaceutical composition comprising thereof, is used in method for improving at least one clinical parameter of Cystic Fibrosis. In some embodiments, an ASO as disclosed and as described hereinabove, or a pharmaceutical composition comprising thereof, is used in treating of CF.

### Kit

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation.

In some embodiments, the kit of the invention comprises: at least one ASO; and at least one of: at least one CFTR modifier; or at least one CF drug, wherein the ASO is selected from SEQ ID Nos.: 4-14, and 19-30, and wherein the CFTR modifier is selected from: CFTR potentiator, CFTR corrector, and CFTR amplifier.

In some embodiments, the CF drug is an antibiotic drug, a bronchodilator, a corticosteroid, or any combination thereof.

Types and doses of CF drugs, such as an antibiotic, a bronchodilator, and a corticosteroid, would be apparent to one of ordinary skill in the art. Non-limiting examples of CF drugs, such as antibiotics include, but are not limited to, cloxacillin, dicloxacillin, cephalosporin, trimethoprim, sulfamethoxazole, erythromycin, amoxicillin, clavulanate, ampicillin, tetracycline, linezolid, tobramycin or aztreonam lysine, fluoroquinolone, gentamicin, and monobactam with antipseudomonal activity.

In some embodiments, the components of the kit disclosed above are sterile. As used herein, the term “sterile” refers to a state of being free from biological contaminants. Any method of sterilization is applicable and would be apparent to one of ordinary skill in the art.

In some embodiments, the components of the kit are packaged within a container.

In some embodiments, the container is made of a material selected from the group consisting of thin-walled film or plastic (transparent or opaque), paperboard-based, foil, rigid plastic, metal (e.g., aluminum), glass, etc.

In some embodiments, the content of the kit is packaged, as described below, to allow for storage of the components until they are needed.

In some embodiments, some or all components of the kit may be packaged in suitable packaging to maintain sterility.

In some embodiments, the components of the kit are stored in separate containers within the main kit containment element e.g., box or analogous structure, may or may not be an airtight container, e.g., to further preserve the sterility of some or all of the components of the kit.

In some embodiments, the instructions may be recorded on a suitable recording medium or substrate. For example, the instructions may be printed on a substrate, such as paper or plastic, etc.

In some embodiments, the instructions may be present in the kit as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Method of Production

According to some embodiments, a method for producing a compound suitable for treating CF is provided.

In some embodiments, the method comprises obtaining a compound that binds to exon 24 of the CFTR pre-mRNA. In some embodiments, the method comprises assaying the skipping of exon 24 of the CFTR pre-mRNA in the presence of the obtained compound. In some embodiments, the method comprises selecting at least one compound that induces the exclusion of exon 24 from the CFTR pre-mRNA.

In some embodiments, the method comprises obtaining a compound that binds to exon 24 of the CFTR pre-mRNA, assaying the skipping of exon 24 of the CFTR pre-mRNA in the presence of the obtained compound, and selecting at least one compound that induces the exclusion of exon 24 from the CFTR pre-mRNA, thereby producing a compound suitable for treating CF.

In some embodiments, the method comprises obtaining a compound that binds to exon 23 of the CFTR pre-mRNA. In some embodiments, the method comprises assaying the skipping of exon 23 of the CFTR pre-mRNA in the presence of the obtained compound. In some embodiments, the method comprises selecting at least one compound that induces the exclusion of exon 23 from said CFTR pre-mRNA.

In some embodiments, the method comprises obtaining a compound that binds to exon 23 of the CFTR pre-mRNA, assaying the skipping of exon 23 of the CFTR pre-mRNA in the presence of the obtained compound, and selecting at least one compound that induces the exclusion of exon 23 from the CFTR pre-mRNA, thereby producing a compound suitable for treating CF.

In some embodiments, the compound is an ASO. In some embodiments, the ASO is an ASO as disclosed and as described herein.

Methods of assaying exon skipping ware common. Non-limiting examples of such methods include, but are not limited to, PCR, qPCR, gene sequencing, northern-blot, dot-blot, in situ hybridization, or others all of which would be apparent to one of ordinary skill in the art.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising”, indicate the inclusion of any recited integer or group of integers but not the exclusion of any other integer or group of integers.

As used herein, the term “consists essentially of”, or variations such as “consist essentially of” or “consisting essentially of”, as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); “Monoclonal Antibodies: Methods and Protocols”. Vincent Ossipow, Nicolas Fischer. Humana Press (2014); “Monoclonal Antibodies: Methods and Protocols”. Maher Albitar. Springer Science & Business Media (2007), all of which are incorporated by reference. Other general references are provided throughout this document.

### Materials and Methods

### Cell Transfection

HEK cells were transiently transfected with a construct bearing a CFTR transcript having exon 24 completely deleted from it (CFTR del Ex24). Transfection was carried out using Lipofectamine 2000 transfection reagent (Invitrogen) according to the lipofectamine 2000 reagent protocol using the following lipofectamine amounts: 96 well—0.15 μl, 6 well—3 μl, 10 mm plate—15 μl.

### Studies of CFTR Function Using a Membrane Potential Assay

HEK cells transfected with the CFTR del Ex24 construct were grown in 96-well (black, flat bottom; corning) plates. 48 hr post-transfection, CFTR channel function was analyzed using the FLIPR membrane potential assay as previously described (Molinski et al., 2015). Briefly, the cells were loaded with blue membrane potential dye (Molecular Devices), which can detect changes in transmembrane potential. The plate was then read in a fluorescence plate reader (BioTek Synergy H1) for baseline levels followed by CFTR stimulation using the cAMP agonist forskolin (10 μM; Sigma), DMSO vehicle was used as a negative control. CFTR-mediated depolarization of the plasma membrane was detected as an increase in fluorescence and hyperpolarization (or repolarization) as a decrease. To terminate the functional assay, the CFTR inhibitor CFTRinh-172 (10 μM; Cystic Fibrosis Foundation Therapeutics) was added to each well. Changes in transmembrane potential were normalized to the values prior to activation.

### 16HBEge N1303K System Studies

In order to analyze the ability of the ASOs to induce skipping over exon 24 in the presence of the mutation N1303K, the inventors used a cellular system that was developed in the CFFT lab, 16HBEge N1303K. The cellular system is based on an immortalized bronchial epithelial cell line which has endogenous WT CFTR containing all exonic and intronic sequences (16HBE14o-) (Cozens et al.,). 16HBE14o- were genetically engineered using CRISPR-based gene editing to establish an isogenic cell line homozygous for the CFTR N1303K mutation (16HBEge N1303K) (Valley et al.,).

### 16HBEge W1282X System Studies

In order to analyze the ability of the ASOs to induce skipping over exon 23 in the presence of the mutation W1282X, the inventors used a cellular system that was developed in the CFFT lab, 16HBEge W1282X. The cellular system is based on an immortalized bronchial epithelial cell line which has endogenous WT CFTR containing all exonic and intronic sequences (16HBE14o-) (Cozens et al.,). 16HBE14o- were genetically engineered using CRISPR-based gene editing to establish an isogenic cell line homozygous for the CFTR W1282X mutation (16HBEge W1282X) (Valley et al.,).

### Transfection

ASOs were transfected into 16HBEge N1303K cells or 16HBEge W1282X cells using Lipofecatmine 2000 transfection reagent (Invitrogen) according to the lipofectamine 2000 reagent protocol. In each experiment the effect of different ASOs was analyzed in comparison to cells treated with a control ASO.

### RNA Extraction

Twenty-four (24) hrs following transfection, total RNA was extracted from the above-mentioned cells using RNeasy Mini Kit (QIAGEN). RNA concentration was determined using a nanodrop. Complementary DNA (cDNA) synthesis was performed using the High Capacity cDNA Reverse Transcription kit (Applied Biosystems). The cDNA was analyzed by PCR.

### Determine the Ratio Between These Two Transcripts (PCR)

PCR was performed using the Platinum™ SuperFi™ Green PCR Master Mix 12359-10 (Invitrogen). PCR products were then separated on an agarose gel for detection of the correctly and aberrantly spliced transcripts. The gels were exposed to UV light for visualization and the PCR products were recorded.

Quantitative Detection of Correctly and Aberrantly Spliced CFTR Transcripts (qPCR)

Real-time PCR was performed in QuantStudi 3 Real-Time PCR System using TaqMan® Fast Advanced Master Mix (Applied Biosystems) with TaqMan probes specific for transcripts including exon 23 or transcripts without exon 23. The expression level was normalized to the transcript levels of GUSb. Technical duplicates were analyzed for each sample. Analysis was performed using the double delta Ct analysis.

### CFTR Protein Analysis by Western Blot

For protein analysis 16HBEge W1282X cells were transfected with the indicated ASO. Twenty-four (24) h following the transfection proteins were extracted using RIPA buffer and analyzed by immunoblotting with CFTR antibodies. Six (6) % polyacrylamide gels were used for protein separation. The gel was transferred to a nitrocellulose membrane, and antibody hybridization and chemiluminescence were performed according to standard procedures. The primary antibodies used in this analysis were mouse anti CFTR 596 (Cystic Fibrosis Foundation Therapeutics) and rabbit anti Calnexin (Sigma). Horseredish peroxidase (HRP)-conjugated anti-rabbit and anti-mouse secondary antibodies were used (Jackson ImmunoResearch Laboratories).

### Example 1

### CFTR Lacking Exon 24 is Functionally Comparable to the WT CFTR

FLIPR™ (Fluorescence Imaging Plate Reader) is a functional system that allows measuring changes in membrane potential by a fluorescent indicator. FLIPR can be used to test CFTR activation levels when the activation of CFTR is achieved by the addition of Forskolin (FSK) and the specificity for the CFTR channel is verified by the addition of CFTR specific inhibitor (inh-172).

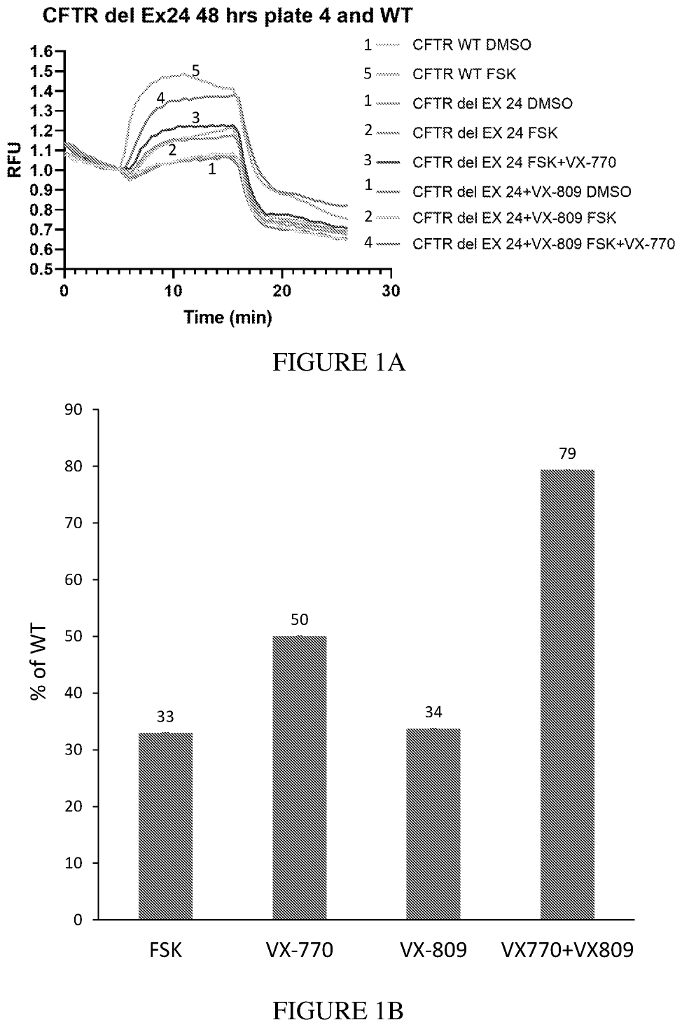

CFTR proteins lacking exon 24 were found to have a residual activity (FIG. 1A). The addition of a potentiator (VX-770) increased the channel activation (50% of WT; FIG. 1B). Moreover, the addition of a corrector (VX-809) and potentiator (VX-770) significantly augmented channel activity (80% of WT; FIG. 1B). Thus, induced skipping of exon 24 which results in CFTR mRNA lacking this exon, provides a CFTR protein functionally comparable to the WT CFTR and, therefore, can be directed to treating of CF.

### Example 2

### ASOs Induce Skipping of Exon 24

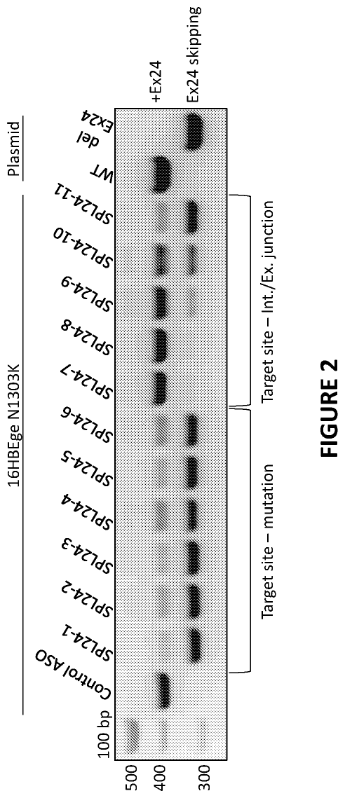

In order to analyze the ability of ASOs to induce skipping over exon 24 in the presence of the mutation N1303K, the inventors used the 16HBEge N1303K cellular system and various ASO, some of which complementary to (e.g., targeting) the exon-intron junction, and some targeting the specific mutation site (i.e. on-mutation ASOs). Surprisingly, the inventors have shown that all of the examined on-mutation ASOs were found to effectively induce skipping of exon 24 (FIG. 2). Further, in most cases, ASOs targeting the exon-intron junction were found to be substantially less reliable and effective in inducing exon 24 skipping, with only a few exon-intron ASOs inducing a comparable exon skipping (FIG. 2).

### Example 3

### ASOs Targeting the W1282X Mutation Site Induce Exon 23 Skipping

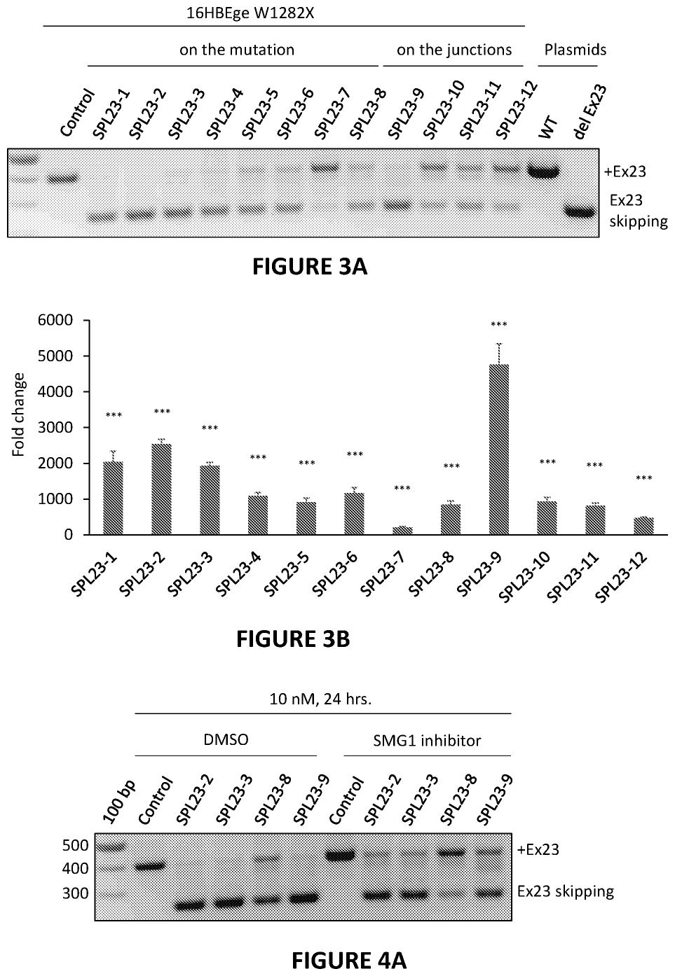

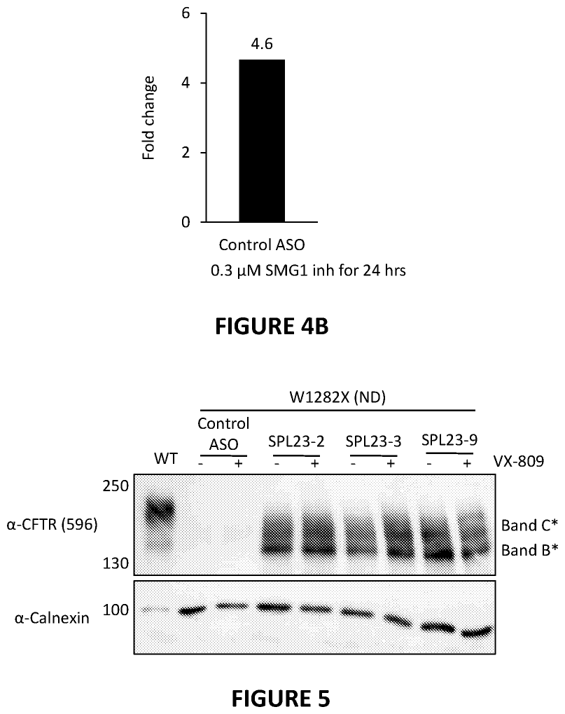

ASOs complementary to a mutated W1282X encoding sequence were found to effectively induce exon 23 skipping (FIG. 3). This effect was found to be highly significant under NMD inhibition with the SMG1 inhibitor (FIG. 4). Cells carrying the W1282X mutation showed no CFTR protein and/or activity. In contrast, the introduction of ASOs that are specifically complementary to the mutated exon 23, induced the exclusion of this exon and lead to a significant production level of a mature and deleted CFTR protein (FIG. 5).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-44. (canceled)

45. A method for treating cystic fibrosis (CF) in a subject in need thereof, comprising administering to said subject a therapeutically effective amount of at least one synthetic antisense oligonucleotide (ASO) that targets a CF-conferring mutation located in exon 24 of the cystic fibrosis transmembrane conductance regulator (CFTR) pre-mRNA, thereby treating CF in the subject.

46. The method of claim 45, wherein said ASO comprises 14 to 25 bases or 17 to 22 bases.

47. The method of claim 45, wherein said ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

48. The method of claim 45, wherein said ASO has at least 75% complementarity to:
a. a sequence consisting of: SEQ ID NO: 1, SEQ ID NO: 15, or both; or
b. a sequence consisting of SEQ ID NO: 2.

49. The method of claim 45, wherein said ASO has at least 80% complementarity to any one of: SEQ ID NO: 1, SEQ ID NO: 15, SEQ ID NO: 2; and SEQ ID NO: 3.

50. The method of claim 45, wherein said ASO comprises 3 mismatched bases at most, compared to a sequence selected from the group consisting of: SEQ ID Nos.: 1-3 and 15.

51. The method of claim 50, wherein one mismatched base at most of said 3 mismatched bases is (a) located not more than 3 bases from the 5′ prime end of said ASO or (b) located not more than 3 bases from the 3′ prime end of said ASO.

52. The method of claim 45, wherein said ASO comprises a cytosine complementary to a guanine located at position 336 of said SEQ ID NO: 1, position 136 of said SEQ ID NO: 2, or position 36 of said SEQ ID NO: 3.

53. The method of claim 52, wherein said ASO comprises 4 to 18 nucleotides upstream to said cytosine.

54. The method of claim 45, wherein said ASO comprises:
(SEQ ID NO: 4)
CCAACUUUUUUCUAAAUGUUCC;
(SEQ ID NO: 5)
UCCAACUUUUUUCUAAAUGU;
(SEQ ID NO: 6)
GGAUCCAACUUUUUUCUAAAUG;
(SEQ ID NO: 7)
GAUCCAACUUUUUUCUAA;
(SEQ ID NO: 8)
CAUAGGGAUCCAACUUUUUUC;
or
(SEQ ID NO: 9)
CAUAGGGAUCCAACUUUUU.

55. The method of claim 45, wherein said subject comprises at least one mutation selected from the group consisting of: N1303K, W1282X, 4006delA, 4010del4, 4015delA, 4016insT, G1298A, T1299I, 4040delA, 4041 4046del6insTGT, 4048insCC, Q1313X, CFTRdele21, G1244E, T1246I, 3876delA, 3878delG, S1251N, L1254X, S1255P, S1255X, 3905insT, D1270N, R1283M, and Q1291R, wherein said X denotes translation termination.

56. The method of claim 45, wherein said treating comprises improving at least one clinical parameter of CF selected from the group consisting of: lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, and the need for antibiotic therapy for sinopulmonary signs or symptoms.

57. The method of claim 45, further comprising administering to said subject a therapeutically effective amount of one or more CFTR modifiers selected from the group consisting of: potentiator, corrector, translational read-through agent, and amplifier.

58. The method of claim 57, wherein said modifier is ivacaftor, lumacaftor, tezacaftor, VX-659, VX-445, VX-152, VX-440, or any combination thereof.

59. The method of claim 45, wherein said composition comprising an ASO is administered by inhalation.

60. A composition comprising an ASO that induces splicing activity of exon 24 of CFTR pre-mRNA comprising 14 to 25 bases or 17 to 22 bases and having at least 80% complementarity to said CFTR pre-mRNA.

61. The composition of claim 61, wherein said ASO comprises a cytosine complementary to a guanine located at: position 336 of said SEQ ID NO: 1, position 136 of said SEQ ID NO: 2, or position 36 of said SEQ ID NO: 3.

62. The composition of claim 62, wherein said ASO comprises 4 to 18 nucleotides upstream to said cytosine.

63. The composition of claim 61, wherein said ASO comprises:
(SEQ ID NO: 4)
CCAACUUUUUUCUAAAUGUUCC;
(SEQ ID NO: 5)
UCCAACUUUUUUCUAAAUGU;
(SEQ ID NO: 6)
GGAUCCAACUUUUUUCUAAAUG;
(SEQ ID NO: 7)
GAUCCAACUUUUUUCUAA;
(SEQ ID NO: 8)
CAUAGGGAUCCAACUUUUUUC;
or
(SEQ ID NO: 9)
CAUAGGGAUCCAACUUUUU.

64. The composition of claim 61, wherein said ASO comprises a chemically modified backbone comprising: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

65. The composition of claim 61, further comprising a pharmaceutically acceptable carrier.

66. The composition of claim 61, formulated for administration via inhalation.

67. A kit comprising at least one ASO that targets a CF-conferring mutation located in exon 24 of CFTR pre-mRNA and:
a. at least one CFTR modifier comprising CFTR potentiator, CFTR corrector, Translational Read-Through agent, and CFTR amplifier; or
b. at least one CF drug comprising an antibiotic drug, a bronchodilator, a corticosteroid, or any combination thereof.

68. The kit of claim 68, wherein said at least one ASO comprises a sequence selected from the group consisting of SEQ ID Nos.: 4-14.

69. The kit of claim 68, wherein said CFTR modifier comprises ivacaftor, lumacaftor, tezacaftor, elexacaftor, VX-659, VX-152, VX-440, VX-371, or any combination thereof.

70. A method for producing a compound suitable for treating CF, the method comprising: obtaining a compound that binds to exon 24 of the CFTR pre-mRNA, assaying the skipping of exon 24 of the CFTR pre-mRNA in the presence of said obtained compound, and selecting at least one compound that induces the exclusion of exon 24 from said CFTR pre-mRNA, thereby producing a compound suitable for treating CF.

71. The method of claim 70, wherein said compound is an ASO.
